(19) United States
(12) Patent Application Publication
(10) Pub. No.: US 2025/0090020 A1
(43) Pub. Date: Mar. 20, 2025

(54) OPHTHALMIC VIEWING SYSTEMS

(71) Applicant: Alcon Inc., Fribourg (CH)

(72) Inventors: Patrick TERRY, Goleta, CA (US); Sumit PALIWAL, Lake Forest, CA (US); Mark Andrew ZIELKE, Lake Forest, CA (US); Lance NOLLER, Lake Forest, CA (US); Nanhong LOU, Southlake, TX (US)

(21) Appl. No.: 18/830,318

(22) Filed: Sep. 10, 2024

Related U.S. Application Data
(60) Provisional application No. 63/582,661, filed on Sep. 14, 2023.

(51) Int. Cl. A61B 3/117; A61B 3/00; A61B 3/103; A61B 3/13; A61B 3/15

(52) U.S. Cl. CPC A61B 3/117; A61B 3/0025; A61B 3/0033; A61B 3/103; A61B 3/13; A61B 3/152

(57) ABSTRACT

Ophthalmic viewing systems for visualizing structures on and in an eye of a medical patient include an ophthalmic microscope, a gonioscopy device configured to work with the ophthalmic microscope to provide an image of an interior of the patient's eye, and a robotic arm configured to hold the gonioscopy device in a position in relation to the ophthalmic microscope and the patient's eye to provide the image of the eye's interior.

### CROSS-REFERENCE TO RELATED APPLICATIONS

This application claims priority to U.S. Provisional Application No. 63/582,661, filed on Sep. 14, 2023, which is hereby incorporated by reference in its entirety.

### TECHNICAL FIELD

This disclosure relates generally to systems for visualizing internal structures of the eye of a medical patient. These systems may be used, for example but not by way of limitation, during ophthalmic surgical procedures, including the placement of intraocular stents or other drainage devices for relieving intraocular pressure associated with glaucoma. Such systems maybe also be used during other situations in which visualization of the eye's internal structures is necessary or desired.

### BACKGROUND

Glaucoma is a disease of the eye, frequently related to excess pressure within the eye's interior. High pressures within the eye can damage the optic nerve, which can lead in turn to worsening vision and even blindness. Glaucoma is one of the most frequent causes of blindness in patients over the age of 60.

Mild glaucoma is sometimes treated through topical applications of appropriate medications. More severe glaucoma may frequently require surgery. These surgeries can include canaloplasty, trabeculectomy, and the placement of implants to increase the drainage of excess fluid from the eye's interior chamber. These procedures can reduce fluid pressure fluid inside the eye, and hence the unwanted and damaging overpressure that might otherwise be applied to the optic nerve.

In some relatively new surgical techniques, called minimally invasive glaucoma surgeries (MIGS), an intraocular micro-stent may be placed at a target site on an interior surface inside the eye. A properly placed micro-stent enhances the travel of intraocular fluid, for example, out of the eye, through the trabecular meshwork, and into Schlemm's canal. Alternatively, certain MIGs procedures may rely on small incisions (and/or excisions) of intraocular tissues for facilitating improved aqueous outflow into the Schlemm's canal, or dilation of the Schlemm's canal via a viscoelastic.

The effectiveness of such surgeries depends, though, on the proper placement of intraocular incisions and/or the proper placement of the micro-stent drainage device. This frequently depends in turn on the effectiveness with which the target site for placement of the intraocular incision or micro-stent can be visualized by the surgeon.

In many cases, the regions of most interest within a patient's eye may be entirely impossible to see without additional assistance. FIGS. 1A and 1B depict two partial cross-sectional views of a patient's eye 100 illustrating this situation. In FIG. 1A, light 101 is reflected from the trabecular meshwork 103 of a patient's eye 105. In most normal eyes, this light undergoes “total internal reflection” at the boundary between the eye's exterior surface 107 and outside air. A view of the trabecular meshwork 103 may thus be partially or even entirely unavailable to an external viewer. Unfortunately, it is precisely at this trabecular meshwork where glaucoma stents are most often placed, and where other similar procedures are frequently performed.

To overcome this limitation, surgeons (or other medical professionals) often use handheld gonioscopy devices, which include prisms, mirrors or combinations of both, to redirect light from inside the eye so that it can be seen from the eye's exterior. In FIG. 1B, a gonioscopy device 110 is placed over the patient's eye 100. With saline occupying the gap 112 between the device 110 and the eye's exterior surface 107, the gonioscopy device 110 directs the light outward where it can be viewed by the device's user.

Conventionally, such gonioscopy devices 110 are held manually in place, which occupies one of the user's hands and which can be difficult and fatiguing to a surgeon or other user.

In addition, where gonioscopy devices such as mirrors are used, the image available to the user may be inverted in comparison to the patient's eye's true configuration. A surgeon must then “de-invert” the image mentally at the same time he or she is using one or more handheld surgical tools to perform very delicate and exacting procedures on fine structures inside and towards the back of the patient's eyeball.

Although experienced and skilled surgeons can overcome these challenges to a considerable extent, such is not easy. Some estimates are, for example, that perhaps 30 percent of glaucoma stents may be mislocated, which is likely due in large measure to difficulties in visualizing their placement locations or in keeping the gonioscopy device properly positioned and aligned between the patient's eye and the surgeon's microscope.

There is thus a substantial unmet need in the art for improved systems for better visualization of the eye's internal structures to facilitate internal stenting and other intraocular surgical procedures, as well as in other situations where visualization of the intraocular structures of a patient's eye are necessary or required.

### SUMMARY

Embodiments described in this disclosure relate generally to ophthalmic viewing systems for visualizing structures on and inside an eye of a medical patient. Examples of such systems may include an ophthalmic microscope, a gonioscopy device configured to work with the ophthalmic microscope to provide an image of an interior of the patient's eye, and a robotic arm configured to hold the gonioscopy device in a position in relation to the ophthalmic microscope and the patient's eye to provide the image of the eye's interior.

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIGS. 1A and 1B depict two views illustrating conventional situations involved in visualizing structures and locations inside an eye of a medical patient.

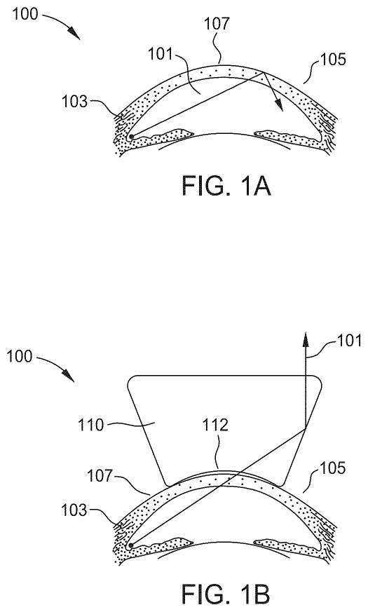

FIG. 2 depicts a perspective view of an ophthalmic viewing system in a surgical operating environment, according to aspects of this disclosure.

FIG. 3 depicts a perspective view of another ophthalmic viewing system in a surgical operating environment, according to aspects of this disclosure.

FIG. 4 depicts a perspective view of another ophthalmic viewing system useful in examining a patient, according to aspects of this disclosure.

FIG. 5 depicts a perspective view of still another ophthalmic viewing system useful in examining a patient, according to aspects of this disclosure.

FIG. 6 depicts details of a robot arm for use with the systems shown in FIGS. 2-5 or similar systems, according to aspects of this disclosure.

FIG. 7 is a block diagram schematically illustrating elements of the systems according to aspects of this disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Various examples will now be described more fully with reference to the accompanying drawings. Systems like those disclosed here may, however, be embodied in various different forms and should not be construed as limited to the examples set forth here.

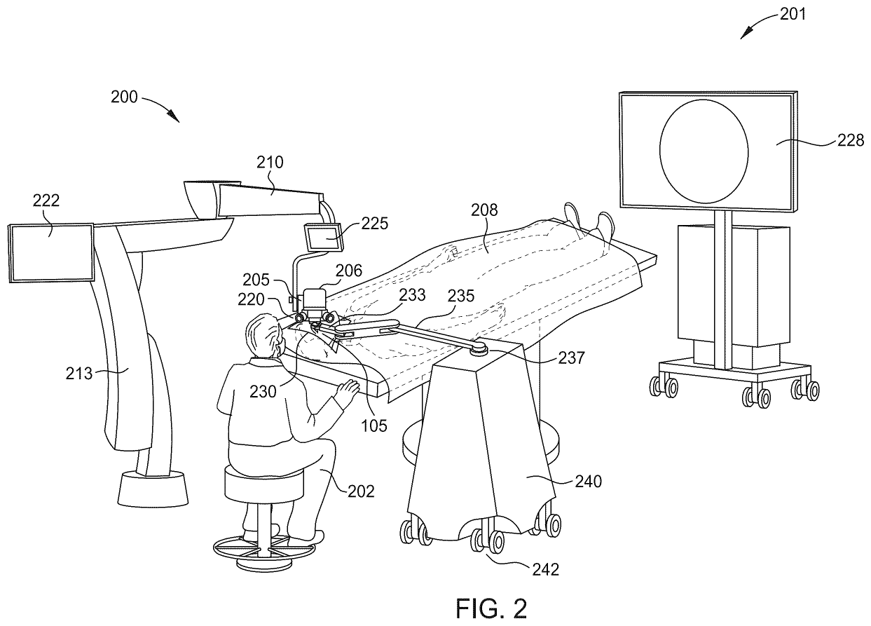

FIG. 2 depicts an ophthalmic viewing system 200 in a surgical operating environment 201 in which a surgeon 202 uses an ophthalmic microscope 205 to visualize structures on and in an eye 105 of a medical patient 208 undergoing a surgery. The microscope 205 is supported on, in this illustration, an adjustable overhead arm 210 of a microscope support pedestal 213.

The patient 208 is supported on an operating table 215. The ophthalmic microscope 205 is movable with the supporting arm 210 in three dimensions so that the surgeon 202 can position the ophthalmic microscope 205 as desired with respect to the eye 105 of the patient 208. The surgeon 202 may in some cases simply grasp the microscope 205 or the arm 210 to manually move the microscope 205 manually into position. In other cases, movement of the microscope 205, arm 210 and/or microscope support pedestal 213 may be performed by stepper motors, servo motors, or similar electro-mechanical actuators at the direction of a controller. For example, in certain embodiments, the microscope 205 may be supported and held by a microscope-supporting robotic arm 210 and/or microscope support pedestal 213, which can be automated and controllable to move the microscope 205 in pre-programmed ways or according to commands inputted by the surgeon 202 or others in the course of the surgery. Generally, the position of the microscope 205, arm 210 and/or microscope support pedestal 213 may be lockable.

In certain embodiments, the ophthalmic microscope 205 comprises a high resolution, high contrast stereo viewing surgical microscope. The ophthalmic microscope 205 will often include a single monocular or dual binocular eyepieces 220, through which the surgeon 202 will have an optically magnified view of the relevant eye structures that the surgeon 202 will need to see to accomplish a given surgery.

The ophthalmic microscope 205 may further include one or more relay lenses, magnifying/focusing optics, objective lenses, and other surgical viewing optics. Generally, the ophthalmic microscope 205 may include any suitable optical or electronic components for providing a view of the patient's eye 105 to the surgeon 202.

The ophthalmic microscope 205 will also frequently include, or be in operably coupled with, an imaging unit 206 comprising one or more cameras or similar electronic visualization/imaging apparatus, sometimes instead of but frequently in conjunction with the optical eyepiece 220. The imaging unit 206 can be used to relay an image of the eye 105 onto one or more display screens 222, 225, or 228, for use by the surgeon 202 or one or more assistants. In certain embodiments, the imaging unit 206 may comprise an OCT system or the like.

The locations of the relevant structures of the eye 105, such as an anterior chamber angle and other peripheral structures, and the optical refraction characteristics of the eye 105 itself, are such that the required views of locations of interest to the surgeon 202 often cannot be had directly with the ophthalmic microscope 205 and/or imaging unit 206 alone.

Typically, the surgeon 202 or an assistant must use a gonioscopy device 230 to obtain the desired view. The gonioscopy device 230 may generally include one or more lenses, prisms, and/or mirrors configured to refract or reflect light from the eye's structures into the microscope 205 or the surgeon's own eye so that the surgeon 202 can see as required to perform the surgery. In certain examples, the gonioscopy device 230 comprises a direct gonioscopy device, such as a Koeppe-, Barkan-, Wurst-, Swan-Jacob-, or Richardson-type goniolens. In certain examples, the gonioscopy device 230 comprises an indirect gonioscopy device, such as a Posner-, Sussman-, Zeiss-, or Goldmann-type goniolens.

In the ophthalmic viewing system 200 of FIG. 2, the gonioscopy device 230 is held and supported at a first, distal end 233 of a gonioscopy support arm 235, which is in turn held and supported at a second, proximal end 237 on a cart 240. The cart 240 has wheels 242 that allow the cart 240 to be moved around the operating environment 201 and positioned as desired.

The gonioscopy support arm 235 can, in some cases, be a simple, non-motorized device configured to hold the gonioscopy device 230 in a fixed location after the surgeon 202 has moved it manually into place.

In more sophisticated systems, the gonioscopy support arm 235 can include a robotic arm of various degrees of complexity. Such a robotic arm would typically include multiple joints and links, with controllable stepper or servo motors (or similar electro-mechanical actuators) at the joints between the links, and operable to move an end effector at a distal end of the support arm 235 into position according to pre-programmed instructions, real-time command issued by a user, or combinations of those two.

Using the gonioscopy support arm 235, frequently including a motorized robotic arm, to move and hold the gonioscopy device 230 at desired locations between the patient's eye 105 in combination with the ophthalmic microscope 205, frees at least one of the surgeon's hands for other, better uses. Supporting the gonioscopy device 230 on the gonioscopy support arm 235 can also be expected to reduce fatigue, perhaps provide a more stable or finely controllable view, and otherwise help the surgeon 202 to perform the many difficult tasks that ophthalmic surgeries may require, which can in turn be expected to yield better surgical outcomes.

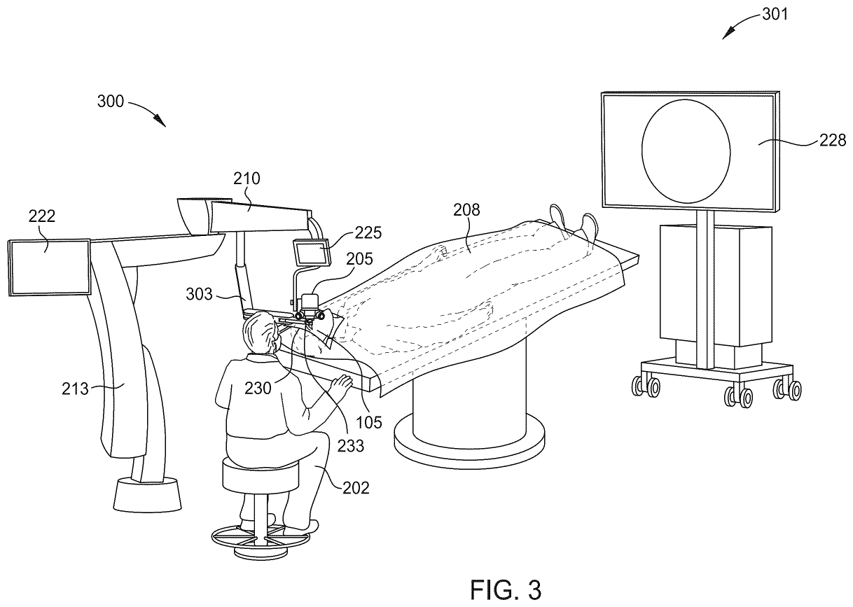

FIG. 3 depicts another ophthalmic viewing system 300 in a surgical operating environment 301. This system 300 shares several elements in common with the ophthalmic viewing system 200 of FIG. 2. The system 300 of FIG. 3 includes, for example, the ophthalmic microscope 205 and imaging unit 206, the overhead arm 210 and support pedestal 213 for supporting the microscope 205 and imaging unit 206 over the patient 208 on operating table 215, and several display screens 222, 225, and 228 for displaying images captured by the microscope 205 and/or imaging unit 206. The microscope 205 and imaging unit 206 may, moreover, be mounted on a microscope supporting robotic support arm, generally as described above in connection with FIG. 2.

In this second surgical system 300, though, an alternative gonioscopy support arm 303 is mounted at its proximal end 305 to the same overhead arm 210 that supports the ophthalmic microscope 205 that the surgeon 202 uses to perform the surgery, in contrast to the system 200 of FIG. 2, in which the gonioscopy support arm 235 is mounted on the wheeled cart 240. In some embodiments, the gonioscopy support arm 303 may be mounted directly to the microscope 205 and/or imaging unit 206.

As with the system 200 of FIG. 2, the gonioscopy support arm 303 in FIG. 3 may in some cases be a simple, non-motorized device that the surgeon 202 can move manually as desired. In other cases, the gonioscopy support arm 303 will be a robotic arm that includes motorized joints and links, with varying degrees of complexity and freedom, controllable by external controls and software to perform various maneuvers to move and position the gonioscopy device in ways helpful to the surgeon 202 in performing various ophthalmological procedures.

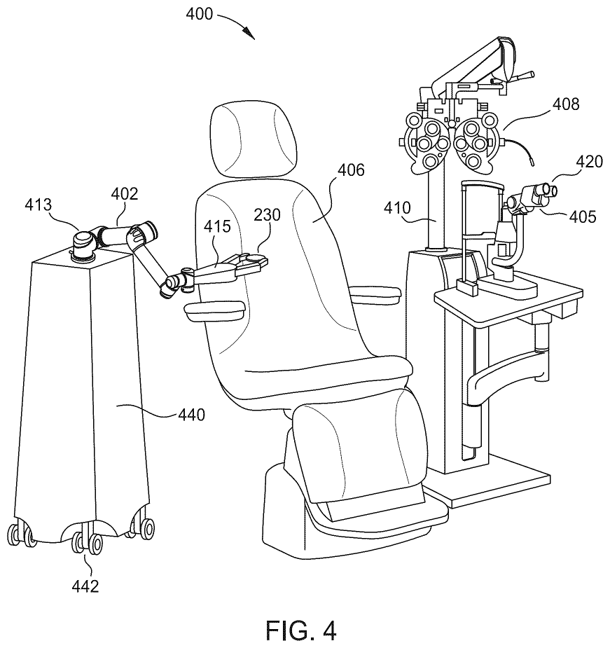

FIG. 4 depicts another ophthalmic viewing system 400 including a gonioscopy support arm 402 configured to hold and allow movement of a gonioscopy device 230 useful performing an eye examination. In FIG. 4, though, the system 400 is shown in connection with an examination chair 406 and other equipment such as one might find in an optometric examination room, rather than the more dedicated surgical context implied in the previous figures. To illustrate this, FIG. 4 includes additional devices including a phoropter 408, a device routinely used by eye care professionals performing a variety of non-surgical examinations, and therefore commonly present where those examinations are typically performed.

FIG. 4 also shows an ophthalmic microscope 405 supported on an equipment stand 410 proximate to the patient's examination chair 406. As with the prior systems 200 and 300 (see FIGS. 2 and 3), a user maneuvers the gonioscopy device 230 into a desired position using the gonioscopy support arm 402. The gonioscopy support arm 402 then holds the gonioscopy device 230 in position while the user looks through eyepieces 420 of the microscope 405 and the gonioscopy device 230 into the locations of interest inside the patient's eye.

Similarly to the system 200 of FIG. 2, in the system 400 of FIG. 4, the gonioscopy support arm 402 is supported at its proximal end 413 on a wheeled cart 440 having wheels 442, with the gonioscopy device 230 held at the support arm's opposite, distal end 415.

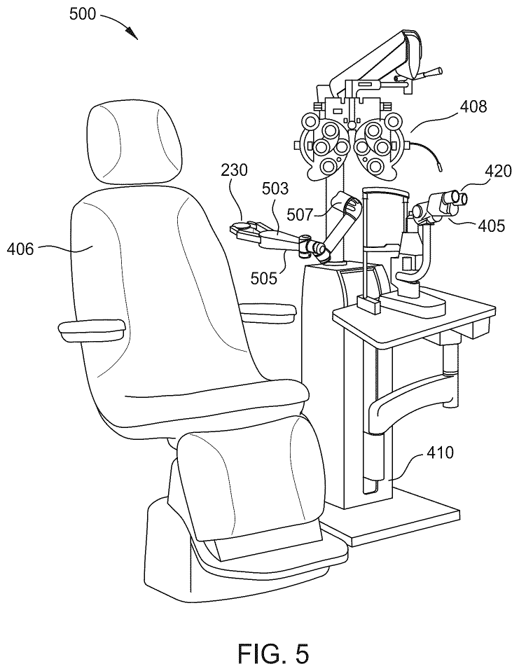

FIG. 5 depicts still another ophthalmic viewing system 500, this one again including a patient examination chair 406 implying use in what may be a primarily non-surgical, examination setting. In this system 500, the gonioscopy device 230 is held at a distal end 503 of a gonioscopy support arm 505 that has a proximal end 507 supported on the equipment stand 410 that holds the examination microscope 405 and perhaps other devices such as the phoropter 408 illustrated in this figure.

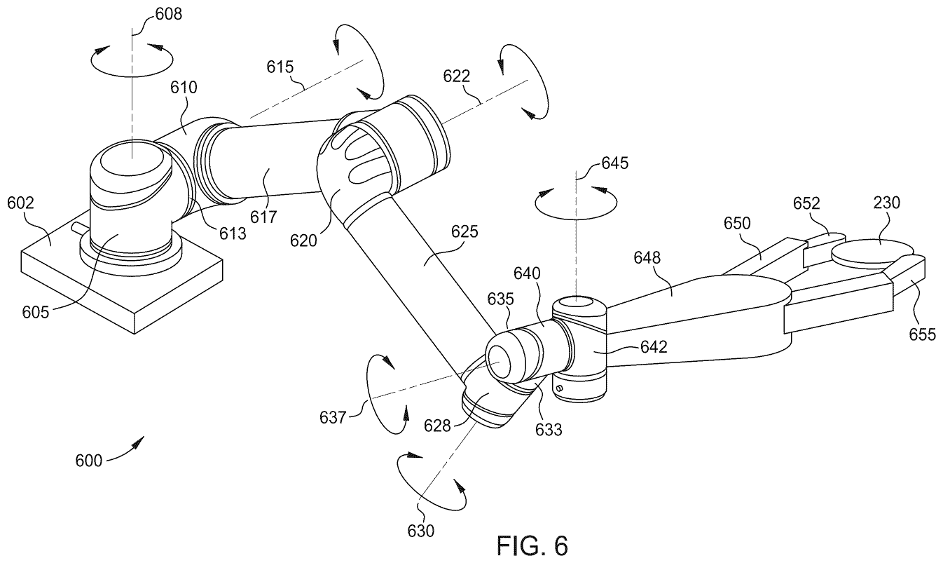

FIG. 6 depicts details of a relatively sophisticated robotic arm 600 that might find use in any of the systems 200, 300, 400, or 500 shown in FIGS. 2-5.

The robotic arm 600 of FIG. 6 includes a support base 602 that may, in some systems, be configured to mount to a movable cart, a fixed support structure, or another device configured to bring the arm 600 into proximity to a patient. The robotic arm 600 further includes a first arm joint 605 that is rotatable with respect to the support base 602 around a first rotation axis 608.

A second arm joint 610 is fixed to the first arm joint 605 via a first link 613. The second arm joint 610 is rotatable with respect to the first arm joint 605 and the first link 613 around a second rotation axis 615. In certain embodiments, the second rotation axis 615 is perpendicular to the first rotation axis 608.

A second link 617 attaches a third arm joint 620 to the second arm joint 610. The third arm joint 620 can rotate with respect to the second link 617 around a third rotation axis 622. In certain embodiments, the third rotation axis 622 is parallel with the second rotation axis 615.

A third link 625 extends between the third arm joint 620 and a fourth arm joint 628. The fourth arm joint 628 rotates with respect to the third link 625 around a fourth rotation axis 630. In certain embodiments, the fourth rotation axis 630 is parallel with the second rotation axis 615.

A fourth link 633 joins the fourth arm joint 628 to a fifth arm joint 635. The fifth arm joint 635 rotates with respect to the fourth link 633 around a fifth rotation axis 637. In certain embodiments, the fifth rotation axis 637 is perpendicular to the first rotation axis 608.

A fifth link 640 attached to the fifth arm joint 635 carries a sixth arm joint 642, which rotates with respect to the fifth link 640 around a sixth axis of rotation 645. In certain embodiments, the sixth rotation axis 645 is perpendicular to the fifth rotation axis 637. The sixth arm joint 642 carries a sixth and final link 648. In this embodiment, a working element in the form of a gripper 650 extends from the sixth link 648, in a direction perpendicular to the arm's sixth axis of rotation 645.

A robotic arm 600 of this general type can in some systems be controlled with foot pedals for hands-free positioning. Such controls may advantageously provide for movement in the three major perpendicular x, y, and z movement axes.

In some embodiments, the robotic arm 600 may include internal force and torque sensors to detect forces applied to the arm by its users. In response to the detection of such forces and torques by the sensors, countervailing commands will be sent to the arm's internal servo controls, thereby moving the arm into the user's desired positions.

In still other embodiments, moreover, the arm 600 may be provided with a camera or similar apparatus for tracking the location of the working end of the arm in relation to the patient's eye, and maintaining the arm's position accordingly.

The gripper 650 includes gripping elements 652 and 655 operable to grip and release small objects including a variety of gonioscopy devices useful for performing ophthalmic and optometric procedures, alone or in conjunction with a variety of microscopes and other instruments. The gripper may thus be configured to receive, to hold, and to release one or more gonioscopy devices. In particular configurations, the gripping elements 652 and 655 can be powered and operable under electronic and mechanical control to open and close to receive, hold, and release the gonioscopy device. In other configurations the gripping elements 652 and 655 might simply be spring-loaded, so that they could be moved apart by a user to receive the gonioscopy device 230 and then returned by spring force to grip the device in place. In still other configurations the gonioscopy device 230 might be fixed permanently in place, with the gripping elements not moveable in normal use, or absent entirely with the device 230 secured directly to the final joint 642 or link 648 of the arm 600. Use of movable grippers may frequently be advantageous in allowing the use of a wide range of conventional gonioscopy devices of various types, in accordance with users' existing preferences and previous experience.

Apparatus included in the robotic arm 600, or in conjunction with the grippers or other means for holding the gonioscopy device 230, may advantageously include mechanisms for rotating the gonioscopy device 230 about at least two perpendicular rotational axes (e.g., roll and pitch mechanisms), so that the gonioscopy device 230 can be aligned properly and in good contact with the eye's surface.

It should be noted that the configurations of the robotic arm 600 in FIG. 6, and various arms in the other drawings in this disclosure, are only exemplary; the exact configuration of those arms can vary considerably in any given system. It may generally be preferred, though, that the robotic arm include elements providing movement with at least six degrees of freedom. One commercially available robotic arm of the general type shown in FIG. 6 is a Model UR5e robotic arm supplied by Universal Robots, a robotic equipment manufacturer with its headquarters in Odense, Denmark (https://www.universal-robots.com).

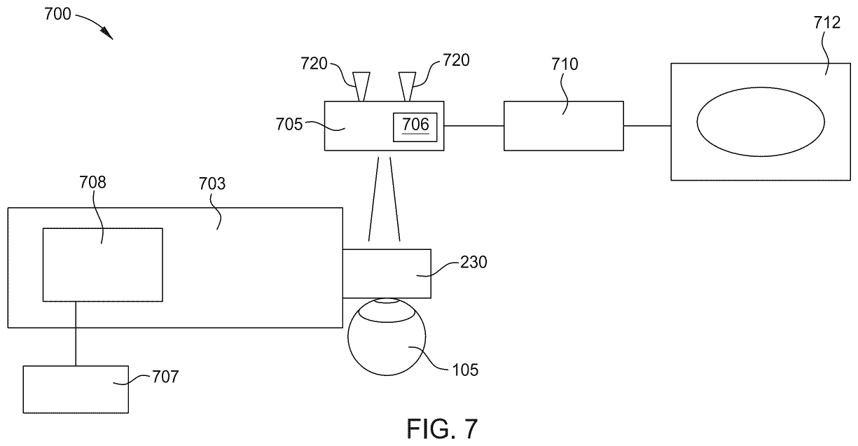

FIG. 7 is a block diagram illustrating elements of a system 700 according to aspects of this disclosure, used by a surgeon or another person to view locations inside an eye 105 of a medical patient. Such a system 700 may include a microscope 705, with one or two eyepieces 720 for direct monocular or binocular viewing.

Light from the patient's eye 105 enters the microscope 705 via a gonioscopy device 230, which, as described above, counters the complete reflection that would otherwise occur at the eye's surface, thereby allowing viewing of the eye's internal structures with the microscope 205.

The gonioscopy device 230 can be moved into and held in an appropriate position with respect to the eye 105 by a robotic arm 703. The robotic arm 703 may include servo motors or similar motion control apparatus 708 for controlling the arm's movements. Some configurations will include external motion control input apparatus 707, which allow the system's users to issue commands controlling the arm's movements. Particular external motion control input apparatus 707 may include force torque (FT) sensors responsive to forces and torques that users apply to the robotic arm 703, foot pedal systems, joysticks, keyboards, or other appropriate control devices by which users might convey desired movement commands to the arm 703. Some robotic arms may be provided with force torque sensors operable to respond to the detection of forces or torques applied to elements of the robotic arm above predetermined acceptable limits. Such configurations can help to prevent damage to the patient's eye by preventing the imposition on the eye of unacceptably high forces by the robotic arm. The motion control apparatus then operates the robot arm in accordance with the user's commands to provide the desired movements.

In many cases, the system's microscope 705 may be operably coupled with imaging unit 706 comprising a camera or other visualization/imaging device for electronic capture and transmission of images from the microscope 705 for external display. Those systems will frequently include electronic image processing apparatus 710 in communication with the imaging unit 706 for processing the images for display on one or more video monitors or other visual display apparatus 712 in communication with the electronic image processing apparatus 710.

The image processing apparatus 710 may in some systems comprise hardware and software or other processing mechanisms for “de-inverting” and thereafter displaying properly images that would otherwise appear reversed in cases where the gonioscopy device 230 includes one or more image inverting mirrors.

The description above has shown, described, and pointed out various features and configurations as applied in various examples. It should be understood, though, that various omissions, substitutions, and changes in the form and details of the example devices can be made without departing from the spirit of the disclosure. It should be understood as well that various features of the type described here can be utilized in various combinations, with individual features included omitted as desired and appropriate. None of these feature should be regarded as required in any particular combination, unless the description clearly requires otherwise. As will be recognized, the elements and combinations described here can be embodied in various forms, some of which may not provide all of the features and benefits described in this disclosure, as some features can be used or practiced separately from others. The scope of protection must therefore be defined primarily by the appended claims rather than the foregoing description, and the scope of those claims must be read to include the full scope of equivalents to which those claims are rightfully and legally entitled.

1. An ophthalmic viewing system comprising:
an ophthalmic microscope;
a gonioscopy device configured to relay images of an interior periphery of a patient's eye to the ophthalmic microscope; and
a robotic arm configured to hold the gonioscopy device in a position in relation to the ophthalmic microscope and the patient's eye to relay the images of the interior periphery of the patient's eye to the ophthalmic microscope for viewing by a user.

2. The ophthalmic viewing system of claim 1, wherein the robotic arm comprises a gripper configured to receive, to hold, and to release the gonioscopy device.

3. The ophthalmic viewing system of claim 2, wherein the gripper is powered and operable to open and close to receive, hold, and release the gonioscopy device.

4. The ophthalmic viewing system of claim 1, wherein the robotic arm comprises a proximal end mounted to a structure configured to support the ophthalmic microscope.

5. The ophthalmic viewing system of claim 1, wherein the robotic arm comprises a proximal end mounted on a wheeled cart.

6. The ophthalmic viewing system of claim 1, wherein the ophthalmic microscope comprises an electronic visualization apparatus configured to capture and transmit images from the ophthalmic microscope for external display on visual display apparatus.

7. The ophthalmic viewing system of claim 6, wherein the visual display apparatus comprises a video monitor.

8. The ophthalmic viewing system of claim 6, and further comprising electronic image processing apparatus configured to process the images from the ophthalmic microscope before the images are displayed on the visual display apparatus.

9. The ophthalmic viewing system of claim 8, wherein the gonioscopy device comprises a mirror that inverts at least a portion of the image of the interior periphery of the patient's eye, and wherein the electronic image processing apparatus is configured to de-invert the inverted portion of the image.

10. The ophthalmic viewing system of claim 1, and further comprising:
a motion control input apparatus operable to receive from the user commands corresponding to desired movements of the robotic arm and then to operate the robotic arm in accordance with those commands to provide the desired movements.

11. The ophthalmic viewing system of claim 10, wherein the motion control apparatus comprises at least one of force and torque sensors responsive to forces and torques that the user applies to the robotic arm, a joystick, and a foot pedal system.

12. A system comprising:
an ophthalmic or optometric viewing device;
a gonioscopy device configured to relay images of an interior periphery of a patient's eye to the ophthalmic or optometric viewing device; and
an adjustable arm configured to hold the gonioscopy device in a position in relation to the ophthalmic or optometric viewing device and the patient's eye to relay the images of the interior periphery of the patient's eye to the ophthalmic or optometric viewing device for viewing by a user.

13. The system of claim 12, wherein the ophthalmic or optometric viewing device comprises an ophthalmic microscope.

14. The system of claim 12, wherein the ophthalmic or optometric viewing device comprises an optometric examination device.

15. The system of claim 14, wherein the optometric examination device comprises a phoropter.

16. The system of claim 12, wherein the adjustable arm is mounted to the ophthalmic or optometric viewing device for holding the gonioscopy device in the position.

17. The system of claim 12, wherein the adjustable arm is mounted to a movable cart for holding the gonioscopy device in the position.

18. The system of claim 12, wherein the adjustable arm is manually adjustable.

19. The system of claim 12, wherein the adjustable arm is a robotic arm.

20. The system of claim 19, further comprising:
a motion control input apparatus operable to receive from the user commands corresponding to desired movements of the robotic arm and then to operate the robotic arm in accordance with those commands to provide the desired movements.
